(19) United States
(12) Patent Application Publication
(10) Pub. No.: US 2019/0134326 A1
(43) Pub. Date: May 9, 2019

(54) DEVICE AND METHOD FOR IMPROVING COGNITIVE AND FUNCTIONAL ABILITIES USING HYPOXIC AND HYPEROXIC GAS MIXTURES

(71) Applicant: AI MEDIQ S.A., Luxembourg (LU)

(72) Inventors: Oleg S. GLAZACHEV, Moscow (RU); Alexey PLATONENKO, Luxembourg (LU); Galina SPIRINA, Moscow (RU); Petr GOLITSYN, Moscow (RU); Rudolf LIKAR, Klagenfurt (AT); Georg PINTER, Klagenfurt (AT)

(21) Appl. No.: 16/097,283

(22) PCT Filed: May 4, 2017
(86) PCT No.: PCT/EP2017/060693

§ 371 Date: Oct. 29, 2018

(30) Foreign Application Priority Data
May 6, 2016 (JP) 16168560.7

(51) Int. Cl. A61M 16/00; A61B 5/11; A61B 5/1455; A61B 5/00

(52) U.S. Cl. CPC A61M 16/0045; A61B 5/1104; A61B 5/14551; A61B 5/024; A61B 5/486; A61M 2230/205; A61B 5/4088

(57) ABSTRACT

The invention relates to methods and devices for training persons by breathing in hypoxic and hyperoxic gas mixtures by means of a device on the order of a ReOxy or CellAir One device, in particular by elderly persons and especially particularly persons with dementia. The cognitive functions are assessed by means of the Dementia Detection Test (DemTec) and Sunderland Clock Drawing Test (CDT); the functional exercise capacity is ascertained for instance by means of a 6-minute walk test (6MWT).

The invention relates to methods and devices for improving cognitive and functional capabilities by breathing in hypoxic and hyperoxic gas mixtures, in particular by means of an especially embodied hypoxicator (https://en.wikipedia.org/wiki/Hypoxicator), especially preferably by means of a device on the order of a ReOxy device or CellAir One device.

A hypoxicator is a device which is intended for furnishing a stimulus for adapting the heart and circulatory system of an individual by breathing reduced oxygen/hypoxic air and tripping mechanisms for compensation.

A ReOxy device is an especially embodied hypoxicator by means of which patients during treatment can be supplied with personalized doses of a reduced-oxygen (hypoxic) air and gas mixture. Unlike conventional hypoxicators, an especially embodied hypoxicator has a mode of operation in which, in addition to the low-oxygen gases, oxygen-enriched gases are generated at intervals and can be supplied to a person.

From EP 1 721 629 A1, a device and a method for intermittent hypoxic training under biofeedback control is learned. In it, a device is used which is embodied for supplying a person in alternation with oxygen-enriched (hyperoxic) air or conventional air.

The method for improving cognitive and functional capabilities of persons by varying the blood oxygen saturation with oxygen or by varying the oxygen concentration in the gas mixture breathed in includes the following:
(a) (intermittently) breathing in hypoxic and hyperoxic gas mixtures via a face mask;
(b) calculating treatment parameters based on an individual reaction of the person to the variation in the oxygen concentration in the gas mixture breathed in, and measuring the oxygen concentration of a pulse oximeter;
(c) continuous monitoring of the arterial oxygen saturation and heart rate with the aid of a pulse oximeter.

The device for use in the aforementioned methods is capable of generating breathing gas mixtures with a low proportion of oxygen (9-21 vol. %) and an elevated proportion of oxygen (21-95 vol. %), by splitting the open air into nitrogen and oxygen and afterward mixing them in a calculated ratio in order to subsequently supply this gas mixture to the person via a face mask. It is employed in order to improve cognitive and functional capabilities of the person by varying the blood oxygen saturation with oxygen or by varying the oxygen concentration in the gas mixture breathed in, and it includes intermittent breathing in of hypoxic and hyperoxic gas mixtures via a face mask, which is connected by a hose to the device for hypoxic and hyperoxic gas mixtures.

The hypoxic gas mixture for use in the device of the invention and the method of the invention is a gas mixture having an proportion of oxygen between 21 and 9 vol. %, and the hyperoxic gas mixture is a gas mixture having an proportion of oxygen between 21 and 95 vol. %.

The duration of a treatment (session) with the hypoxic gas mixture of the hypoxic interval is calculated based on an individual reaction of the person to the reduction in the oxygen concentration in the gas mixture breathed in. This can happen based on the degree of reduction in the blood oxygen saturation, and the duration is at maximum 1200 seconds.

The duration of treatment with the hyperoxic gas mixture of the hyperoxic interval is calculated on the basis of an individual reaction of the person to the elevation of the oxygen concentration in the gas mixture breathed in, specifically based on the degree of restoration of the blood oxygen saturation; the original values at the time the hypoxic gas mixture was initially supplied are restored. In the event of a rapid rise in the blood oxygen concentration, in particular a rapid blood oxygen saturation after a treatment with the hypoxic gas mixture, the hyperoxic gas mixture can be adjusted overall with a low oxygen concentration and/or the duration of treatment with the hyperoxic gas mixture can be shortened. If the oxygen concentration of the person rises slowly after a treatment with the hyperoxic gas mixture, the oxygen concentration of the hyperoxic gas mixture can be increased, and/or the treatment with the hyperoxic gas mixture can be made longer.

For determining a rapid or slow saturation of the blood, a time frame for observation is set, in which the regeneration of the oxygen saturation after a treatment with a standard hypoxic gas mixture is observed. The standard hypoxic gas mixture preferably has an oxygen concentration of 12 vol. %. Preferably, the observation time frame amounts to 3 minutes. Especially preferably, the standard hyperoxic gas mixture has an proportion of oxygen of 30 vol. %. If a blood oxygen saturation in the observation time frame is not achieved, then a slow increase in the blood oxygen concentration, in particular a slow blood oxygen saturation, may be present.

For generating the hyperoxic and hypoxic gas mixtures, the device of the invention can have a chamber in which a membrane for gas separation is provided, in particular a zeolite membrane. By the partial passage of components of the air through the membrane, two gas flows are formed: an oxygen-enriched first gas flow, and an oxygen-depleted second gas flow. The first flow has a proportion of 50±10 vol. % of oxygen. The second flow has a proportion of 85±3 vol. % of nitrogen. Preferably, the second gas flow is formed such that upon recombination with the first gas flow, it forms conventional air. Both gas flows may, however, also be further enriched with additional oxygen or nitrogen from appropriate gas sources, such as gas bottles.

The second gas flow, in particular the nitrogen, is carried through a gas mixer into a first collection bag. The first gas flow, in particular the oxygen, is also delivered to a second collection bag and carried to the gas mixer via a proportional valve.

The quantity (flow) of oxygen that which is admixed with the nitrogen flow before or at the time of the discharge, is determined such that the bag with breathing gas mixture is brought to a predetermined, set, variable concentration, specifically in each case for both hypoxic and hyperoxic intervals. Gases at the inlet are directed to the gas mixer and the output is controlled by the securing bag.

The session or sessions each last from 10 to 90 minutes. The number of intervals is determined by the preset duration of the session. The number of sessions can vary between 5 and 25.

The device can include a face mask, preferably a nose and mouth mask. The pulse oximeter can include a finger sensor and/or ear sensor.

The cognitive functions are assessed by means of Dementia Detection Test (DemTec) and Sunderland Clock Drawing Test (CDT), but are not limited to this; the functional exercise capacity is ascertained by means of a 6-minute walk test (6MWT) but are not limited to that.

The (intermittent) inhalation of hypoxic and/or hyperoxic gas mixtures by means of a device on the order of a ReOxy device leads to improved cognitive functions and/or improved functional exercise capacity in the patients studied.

In EP 1 721 629 A1, no IHHT training in the sense of the present invention is disclosed. In particular, the oxygen concentration of the hyperoxic gas mixture, the provision of a hypoxic gas mixture, the usage intervals of the individual gas mixtures, and the number of interval repetitions, leading to an enhancement of cognitive capabilities, are not disclosed in this reference.

Unlike that, the device according to the present invention is embodied for furnishing both hypoxic gas mixtures and hyperoxic gas mixtures. They are supplied to a person by the method of the invention in alternation in an interval training (IHHT) process.

The invention will be described in further detail in terms of the following exemplary embodiment:

### Example 1

Burtcher M. et al (Austria) conducted a randomized, stratified (graduated) placebo-controlled double-blind study with 41 patients involved, aged from 64 to 92. They were arbitrarily distributed to the intervention group and the control group. Both groups completed the same multi-modal training; however, while the intervention group additionally received treatments by means of inhaling hypoxic and hyperoxic gas mixtures (hereinafter IHHT), the control group conversely received an air mixture a standard amount oxygen. Changes in the cognitive capability were assessed using the Dementia Detection Test (DemTect) and the Sunderland Clock Drawing Test (CDT). Changes in the functional exercise capacity were measured by means of the total distance of 6-minute walk tests (6MWT).

For the hypoxia group, each IHHT session lasted for up to one hour. The IHHT session parameters (maximal duration of the hypoxia period, maximal duration of the hyperoxia period, hypoxic O₂ concentration were calculated individually, based on the outcomes of the hypoxic test HT (O₂ concentration 12%; duration of hypoxic period 10 minutes maximum, hyperoxic period 3 minutes maximum).

After this test, the participants in the hypoxic group were exposed repeatedly for from 4 to 7 minutes to a hypoxic gas mixture (10-14%) followed by exposure for 2 to 4 minutes to a hyperoxic gas mixture (30%) through a face mask on every treatment day. Each session lasted from 30 to 40 minutes and encompassed from 4 to 8 hypoxy/hyperoxy cycles, depending on the person's individual reaction and condition.

The normal oxic group (control group) completed the program in the same way breathing only normal oxic air (sham treatment).

The researchers conducted IHHT sessions using the ReOxy device for breathing therapy. This device provided the participants with gas mixtures (10-30%), and the arterial oxygen saturation (SaO₂) was monitored at the same time. Both the proportion of SaO₂ and the heart rate were measured with the aid of a pulse oximeter, which sent all the measurement data to the device without the data being visible to the test persons.

During the entire time spent in the geriatric daily clinic, a total of 12-15 hypoxic or sham treatments were conducted, twice to three times a week within from 5 to 7 weeks for both groups.

Regarding the cognitive tests by means of DemTect, the control group exhibited a high significant increase after the IHHT combined with the MIT (+16.7%), while the control group, which did only the MIT exhibited no increase (−0.39%). The CDT showed similar results with a significant increase in the intervention group (+10.7%) while conversely the score of the control group even decreased slightly (−8%). Regarding the functional exercise capacity, both groups showed an increase in total distance in the 6MWT, but in the intervention group the increase was significantly higher than in the control group (+24.1% vs. +410.8%).

### DETAILED DESCRIPTION

More than 46 million people suffer today from dementia because of severe economic and financial burdens. It is extremely likely that this number will rise dramatically by the year 2050, to 131.5 million people. There is still an enormous need for new strategies for prevention and treatment of dementia.

One such strategy can be IHHT. IHHT proved to be a noninvasive, easy-to-use and safe method based on repeated action with a gas mixture having a reduced proportion of oxygen; this action is interrupted by recovery phases from time to time. These recovery phases can be either normoxic (20-21 vol. % O₂, IHT) or hyperoxic (21-95 vol. % O₂, IHHT). The repeated action of the gas mixture with a reduced proportion of oxygen causes an only moderate burden, followed by beneficial adaptations. Burtscher M. Katayama and colleagues showed that the IHT exercise tolerance in patients who suffer from chronic diseases such as heart and circulatory diseases, chronic lung disease, or metabolic diseases, increases. It has also been demonstrated that IHT can prevent oxidative and nitrosative stress, protects against neurodegenerative changes, and even improves cognitive functions in experimental Alzheimer's disease in rats. To our knowledge, Schega L. et al were the first to research the affects of IHT on the cognitive capability of elderly people between 60 and 70 years old. In their pilot study, a strength endurance training program enhanced the cognitive functions, and the additional IHT enhanced this positive effect of the exercises with regard to cognitive capability and quality of life.

A novel form of the hypoxic effect is proposed, which is called intermitted hypoxic/hyperoxic training (IHHT). IHHT is characterized by even more-positive effects than conventional IHT. The standard oxic periods are replaced with moderate hyperoxic periods of 30-40% O₂. This leads to faster recovery from the oxygen depletion. It is asserted in one study with male Wistar rats that the hypoxic-hyperoxic treatment results in a better and faster membrane-stabilizing effect in heart, liver and brain cells in comparison to IHT. A current study has shown that the IHHT has improved the training achievement in athletes who have overtraining syndrome. The case study by Susta et al ascertained a marked improvement in cardiopulmonary efficiency (heart-lung efficiency) and lactate clearance after 3 weeks of daily IHHT. Glazachev showed that IHHT can improve the exercise tolerance, aerobic capacity, and cardiometabolic profile in patients with NYHA II-III without any exercises at all. None of these studies reports unwanted side effects. IHHT even appears to exceed the positive results of IHT.

The ReOxy device used in the research carried out by Burtcher is described in further detail at www.ReOxy.lu. This device uses the Self-Regulated Treatment (SRT) technology. SRT technology is based on the biofeedback principle; the patient's bodily functions, which are monitored during the entire duration of treatment, determine the treatment parameters. SRT technology is based on highly developed software, which constantly reads out and analyzes data of the built-in pulse oximeter for the sake of adapting the composition of the air mixture administered and its regulation over time. These, that is, the composition and regulation of the air mixture, are based on changes in the patient's vital functions (such as blood oxygen saturation (SpO₂) and heart rate) (www.ReOxy.lu).

The present invention relates in particular to a device for furnishing hypoxic/hyperoxic gas mixtures, having means for continuous measurement of the arterial oxygen saturation and/or heart rate and for ascertaining individual parameters based on the person's individual reaction to the physical effect of the hypoxic/hyperoxic gas mixtures; the device is embodied for generating gas mixtures with a low and an elevated proportion of oxygen by splitting the open air into nitrogen and oxygen and then as a function of the individual reaction mixing them in a calculated ratio; the device can be used for intermittent hypoxic/hyperoxic training (IHHT) of persons to whom the gas mixtures are supplied by means of an apparatus which is connected to the device for hypoxic and hyperoxic air. According to the invention, it is provided that the low proportion of oxygen includes from 8-20 vol. % and the elevated proportion of oxygen includes 21-95 vol. %.

In a refinement of the invention, it is provided that the hypoxic gas mixture includes from 10%-16% oxygen and the hyperoxic air mixture includes from 30%-40% oxygen.

Preferably, it is provided that a face mask is provided, by means of which the hypoxic/hyperoxic gas mixture can be inhaled, and/or that the device has a pulse oximeter, by means of which the arterial oxygen saturation and/or heart rate can be determined; the pulse oximeter can include a finger sensor and/or ear sensor, by means of which the arterial oxygen saturation and/or heart rate can be detected.

Preferably, the device operates on the biofeedback principle. The present invention furthermore relates to a method for furnishing intermittent hypoxic/hyperoxic training (IHHT), including furnishing hyperoxic/hypoxic air to a person using the device of one of claims 1 through 10, in which (a) the arterial oxygen saturation and/or heart rate is measured continuously; (b) individual parameters are ascertained on the basis of an individual reaction of the person; (c1) the oxygen concentration in the hypoxic/hyperoxic air supplied is adapted in accordance with the values ascertained in (b) and/or (c2) the degree of blood oxygen saturation is varied.

Preferably, the duration of the hypoxic interval is calculated such that the restoration of the blood oxygen saturation with oxygen to the saturation values at the beginning of the previous delivery interval is ensured; the training can be repeated, preferably between 5 and 25 times, during a sufficient number of training sessions.

Especially preferably, the duration of the hypoxic interval can amount to a maximum of 1200 seconds; the IHHT training can lead to improved cognitive functions and/or improved functional capacity in the presence of physical stresses.

Preferably, the cognitive capabilities are evaluated with the aid of standardized tests, including but not limited to the DemTect tests and the Sunderland Clock Drawing Test (CDT); the improved functional capacity in the case of physical stresses is evaluated with the aid of standardized function tests, including the 6-minute walk test (6MWT), but not limited to that.

Especially preferably, the hypoxic air mixture can include from 10%-16% and the hyperoxic air mixture can include from 30%-40%.

Furthermore, the invention relates to a method for controlling the operation of the device of the invention, in which method the device is operated in succession and repeatedly in a first mode, in which it generates hypoxic gas, and in a second mode, in which it generates hyperoxic gas; a component of the device measures the arterial oxygen saturation and/or heart rate of a person, and based on the measured values, it dynamically adapts and sets the duration of operation in the first mode.

According to the invention, a feedback loop between a person's measured oxygen saturation and/or heart rate and the duration of the phase in which hypoxic gas or the phase in which hyperoxic gas is delivered to the person can be dynamically adapted and reset upon each repetition of the individual phases.

Especially preferably, the one adaptation or setting of the duration of the phase, that is, lengthening or shortening the phase, can also be set based on oxygen saturation and/or heart rate, even if the phase has already begun.

Preferably, based on the oxygen saturation and/or heart rate of the person, the duration of the second mode is also set.

Especially preferably, the device is operated in the first mode with an oxygen saturation of 10-16 vol. % and in the second mode with an oxygen saturation of 30-40 vol. % and even more preferably with 33-37 vol. % in the second mode.

According to the invention, the following definitions are made:
that the individual reaction can exhibit an individual hypotoxic reaction and an individual hypertoxic reaction;
that the individual hypoxic reaction of the person can exhibit the specific degree of reduction in the blood oxygen saturation because of reducing the oxygen concentration in the gas mixture breathed in, in particular the hypoxic gas mixture; and
that the individual hyperoxic reaction of the person can exhibit the specific degree of restoration of the blood oxygen saturation because of the elevation of the oxygen concentration in the gas mixture breathed in, in particular the hyperoxic gas mixture.

The device and the method of the invention are embodied in a healthy person, that is, who has no cognitive deficiencies, in the context of a training session, in particular a capacity-increasing training session, to improve his cognitive capabilities.

### REFERENCES

1. Basovich S N. Trends in the use of preconditioning to hypoxia for early prevention of future life diseases. Biosci Trends 2013;7:23-32.

2. Burtscher M, Gatterer H, Szubski C, Pierantozzi E, Faulhaber M. Effects of interval hypoxia on exercise tolerance: special focus on patients with CAD or COPD. Sleep Breath. 2009 Aug. 18 PubMed PMID: 19688232.

3. Burtscher M, Pachinger O, Ehrenbourg let al (2004) Intermittent hypoxia increases exercise tolerance in elderly men with and without coronary artery disease. Int J Cardiol 96:247-254

4. Korkushko O, Asanov E, Shatilo V, Makovskaya L. Efficeincy of interval normobaric hypoxische training in elderly subjects. Problems of aging and long living. 2004, 13, Vol. 2,155-161

5. Lei Xi, Serebrovskaya T V. Intermittent hypoxia: from molecular mechanisms to clinical applications. Nova science publishers. 2009.

6. Ischuk V, Shatilov. Intermittent normobaric hypoxia trainings recommendation for elderly ischemic heart disease ptients. Krovoobig ta Gemostaz. 2007, Vol 1: 49-53

7. Ishchuk V. (2007). Safety and efficacy of intermittent normobaric hypoxia training in elderly patients with ischemic heart disease. J. Ukrainian Acad. Med. Sci. 13:374-384.

8. Lyamina N P, Lyamina S V, Senchiknin V N, Mallet R T, Downey H F, Manukhina E B. Normobaric hypoxia conditioning reduces blood pressure and normalizes nitric oxide synthesis in patients with arterial hypertension. J Hypertens 2011:29:226572

9. Schega L., Peter B, Törpel A, Mutschler H, Isermann B, Hamacher D. Effects of intermittent hypoxia on cognitive performance and quality of life in elderly adults: a pilot study. Gerontology. 2013; 59(4):316-23.

10. Shatilo V B, Korkushko O V, Ischuk V A, Downey H F, Serebrovskaya T V. Effects of intermittent hypoxia training on exercise performance, hemodynamics, and ventilation in healthy adults. High Alt Med Biol. 2008 Spring;9(1):43-52. PubMed PMID: 18331220.

11. EP 1 721 629 A1

1. A device for furnishing hypoxic/hyperoxic gas mixtures having means for continuous measurement of the arterial oxygen saturation and/or heart rate as well as for ascertaining individual parameters of the action of hypoxic/hyperoxic gas mixtures on the basis of the person's individual reaction, by means of which breathing mixtures with a low and with an elevated proportion of oxygen can be generated by splitting the atmospheric air into nitrogen and oxygen and subsequently mixing them in a defined ratio, which device can be employed for intermittent hypoxic/hyperoxic training (IHHT), in which the gas mixtures are supplied by means of an apparatus which is connected to the device for hypoxic and hyperoxic air,
characterized in that
the low proportion of oxygen includes from 8-20 vol. % and the elevated proportion of oxygen includes 21-95 vol. %.

2. The device of claim 1,
wherein the hypoxic gas mixture used for the IHHT contains from 10-16 vol. % oxygen and the hyperoxic gas mixture used for the IHHT contains from 21-40 vol. % oxygen.

3. The device of one of claim 1 or 2,
wherein the hypoxic/hyperoxic gas mixture is inhaled by means of a face mask.

4. The device of one of claims 1 through 3,
wherein arterial oxygen saturation and heart rate are measured by means of a pulse oximeter.

5. The device of claim 4,
wherein the pulse oximeter includes a finger sensor and/or ear sensor.

6. The device of one of claims 1 through 5,
wherein the device functions on the biofeedback principle.

7. A method for furnishing intermittent hypoxic/hyperoxic training (IHHT), including the furnishing of hypoxic/hyperoxic air to a person using the device of one of claims 1 through 6,
wherein
(a) the arterial oxygen saturation and/or heart rate is measured continuously;
(b) individual parameters are ascertained on the basis of an individual reaction of the person;
(c1) the oxygen concentration in the hypoxic/hyperoxic air supplied is adapted in accordance with the values ascertained in (b) and/or
(c2) the degree of blood oxygen saturation is varied.

8. The method of claim 7,
wherein the duration of the hyperoxic interval is calculated such that the restoration of the blood oxygen saturation to the physiological normal values is ensured.

9. The method of one of claim 7 or 8,
wherein the training is repeated during a sufficient number of training sessions, preferably between 1 and 25 sessions.

10. The method of one of claims 7 through 9,
wherein the duration of the hyperoxic interval is a maximum of 1200 seconds.

11. The method of one of claims 7 through 10,
wherein the person is an elderly person, in particular a person with poor cognitive capabilities.

12. The method of claim 11,
wherein the IHHT training leads to improved cognitive functions and/or improved functional capacity under physical stresses.

13. The method of claim 12,
wherein the cognitive capabilities are evaluated with the aid of standardized tests, including the Dementia Detection Test and the Sunderland Clock Drawing Test (CDT), but is not limited to these.

14. The method of claim 12,
wherein the improved functional capacity under physical stresses is evaluated with the aid of standardized function tests, including the 6-minute walk test (6MWT), but not limited to that.

15. The method of one of claims 10 through 14,
wherein the hypoxic air mixture includes from 9-20% oxygen and the hyperoxic air mixture includes from 21-40% oxygen.
